(19) United States
(12) Patent Application Publication
(10) Pub. No.: US 2013/0295080 A1
(43) Pub. Date: Nov. 7, 2013

(54) Pharmaceutical composition for treating melasma and preparation method thereof

(71) Applicants: Dejun Sun, Changchun (CN); Jinlong Yin, Changchun (CN); Miaonan Sun, Changchun (CN); Yizhuo Zhao, Changchun (CN); Chunsheng Guo, Changchun (CN); Yanhui Gao, Changchun (CN); Xue Li, Changchun (CN)

(72) Inventors: Dejun Sun, Changchun (CN); Jinlong Yin, Changchun (CN); Miaonan Sun, Changchun (CN); Yizhuo Zhao, Changchun (CN); Chunsheng Guo, Changchun (CN); Yanhui Gao, Changchun (CN); Xue Li, Changchun (CN)

(73) Assignee: Jilin Zixin Pharmaceutical Research Institution LLC, Jilin (CN)

(21) Appl. No.: 13/980,879

(22) PCT Filed: Jul. 15, 2011
(86) PCT No.: PCT/CN11/77177

§ 371 Date: Jul. 20, 2013

(30) Foreign Application Priority Data
Jan. 21, 2011 (CN) 201110023840.6

(51) Int. Cl. A61K 36/8945; A61K 36/752; A61K 36/258; A61K 36/537; A61K 36/076; A61K 36/484; A61K 36/736; A61K 36/185; A61K 36/65; A61K 35/74; A61K 36/534

(52) U.S. Cl. CPC A61K 36/8945; A61K 35/74; A61K 36/752; A61K 36/258; A61K 36/537; A61K 36/534; A61K 36/484; A61K 36/736; A61K 36/185; A61K 36/65; A61K 36/076
USPC 424/123

(57) ABSTRACT

A pharmaceutical composition for treating melasma is provided. The pharmaceutical composition is prepared from ginseng, salvia, mint, orange peel, peony, poria, licorice, peach seed, yam, penthorum chinense and a fermentation conversion solution of fruits and vegetables. A preparation method of the pharmaceutical composition is also provided.

### CROSS REFERENCE OF RELATED APPLICATION

This is a U.S. National Stage under 35 U.S.C 371 of the International Application PCT/CN2011/077177, filed Sep. 15, 2011, which claims priority under 35 U.S.C. 119(a-d) to CN 201110023840.6, filed Jan. 21, 2011.

### BACKGROUND OF THE PRESENT INVENTION

1. Field of Invention

The present invention relates to a pharmaceutical composition for treating melasma and a preparation method thereof.

2. Description of Related Arts

Melasma does harm to a lot of women. The melasma is mainly caused by female endocrine disorders, mental stress, various diseases (liver and kidney function insufficiency, gynecological disease, diabetes, etc) as well as lack of vitamins and external chemical stimulation. For treating the melasma on skin, prevention combined with treatments is mainly provided. Conditioning female endocrine environment, keeping in a good mood, and actively preventing the gynecological diseases are effective methods for preventing the melasma. Specialized medicine such as the effective Grace Age activating protein is also utilized for treating the melasma.

Ginseng: a root of panax of the family Araliaceae. More than 30 kinds of ginseng saponins can be separated out from the ginseng (the ginseng saponins can be divided into three groups: oleanolic acid group, protopanaxdiol group and protopanaxatriol group), and are called ginsenoside-RX (wherein X=0, a1, a2, a3, b1, b2, b3, c, d, e, f, g1, g2, g3, h1, h2, h3, s1, and s2); furthermore, pseudoginsenoside saponin F11 can also be separated out.

Peony: a root of paeonia lactiflora of the family Paeoniaceas of the Class Dicotyledoneae. The peony tastes bitter and sour, and can benefit liver and spleen diseases. The peony cleans blood, nourishes liver, relieves rheumatic symptoms, reduces pain, and arrests sweating. The peony cures thoracic and abdominal flank pain, diarrhea, abdominal pain, spontaneous sweating, cold, fever, irregular menstruation, uterine bleeding and vaginal discharge.

Poria: parasitic fungi on pine roots. The poria tastes sweet and light, has no side effects and benefits heart, lung, spleen, and kidney. The poria also induces diuresis, benefits spleen and stomach, and calms nerve.

Licorice: a root or rhizome of glycyrrhiza uralensis of the family Fabaceae of the Class Dicotyledoneae. The licorice has no side effects, tastes sweet and benefits viscera. The licorice has detoxification, expectorant, analgesic, antispasmodic as well as anti-cancer pharmacological effects. The licorice can be utilized for treating weak stomach and spleen, fatigue, heart palpitations, shortness of breath, cough, sputum, abdominal pain, limbs twin urgent pain, carbuncle swollen sore and drug toxicity.

Yam: a root of dioscorea opposite. The yam is sweet, warm, has no side effects and no toxic, benefits lung, spleen and kidney, and doesn't cause dryness-heat; the yam is good to spleen and stomach, strengthens kidney, vigor, ears and eyes, viscera as well as bones, calms nerve and has anti-aging effects. The yam is mainly utilized for weak spleen and stomach, fatigue, weakness, loss of appetite, diarrhea, weak lung, sputum, cough, weak kidney, weak waist, weak limb, frequent urination, nocturnal emission, premature ejaculation, whitish vaginal discharge, red skin, swelling, obesity and other illnesses.

Mint: an aboveground part of perennial herb of the family Lamiaceae of the genus Mentha, the mint is spicy and cool, benefits lung and liver, relieves rheumatism as well as heat, and cleans toxic. The mint cures cold, headache, red eyes, sore throat, indigestion, mouth sores, toothache, scabies and urticaria.

Salvia: a dried root or rhizome of salvia miltiorrhiza of the family Lamiaceae of the Class Dicotyledoneae. The salvia is bitter and slightly cold. And the salvia benefits heart and liver, invigorates the circulation of blood, regulates menstruation, decreases blood stasis and pain, removes carbuncle, calms nerves, and nourishes blood. The salvia also cures irregular menstruation, amenorrhea, dysmenorrhea, abdominal distension, chest and abdomen pain, arthralgia, sore throat, upset, insomnia; hepatosplenomegaly and angina.

Peach seed: a seed of peach of the rosids plant. The peach seed is bitter, sweet, and has no side effects. And the peach seed benefits heart, liver, and intestine. The peach seed also cures blood stasis, astriction, cough, asthma, amenorrhea, dysmenorrhea, abdominal distension, and dryness of intestine.

Orange peel: peels of orange of the family Rutaceae. The orange peel is spicy, bitter and warm. And the orange peel benefits spleen and lung. The orange peel also treats poor immunity, sputum, dyspepsia, abdominal distention, vomiting, chest tightness due to dampness and weak breath, poor appetite, and loose stools.

Penthorum chienese: a dried aboveground part of penthorum chinense pursh of the family Saxifragaceae of the genus Penthorum. The penthorum chienese is warm, sweet, and non-toxic, and the penthorum chienese has clearing heat, diuretic and urinary elimination, detoxification, blood circulation, strengthening liver and spleen, removing melasma and other effects. The penthorum chienese is mainly utilized for treating melasma, edema, amenorrhea, vaginal bleeding, vaginal discharge, bruises, as well as various types of hepatitis, cholecystitis and fatty liver.

### SUMMARY OF THE PRESENT INVENTION

An object of the present invention is to provide a pharmaceutical composition for treating melasma and a preparation method thereof.

Accordingly, in order to accomplish the above object, the present invention provides a preparation method of a pharmaceutical composition for treating melasma, comprising:

mixing the following four substances for obtaining a mixture, wherein the mixture is the pharmaceutical composition for treating the melasma, wherein the four substances comprises:

A) ethanol extract of ginseng and salvia; B) volatile oil of mint and orange peel; C) aqueous extract of the ginseng, the salvia, the mint, the orange peel, peony, poria, licorice, peach seed, yam and penthorum chienese; D) a fermentation conversion solution of fruits and vegetables;

wherein a method for preparing the A) substance comprises: mixing the ginseng and the salvia, processing reflux extraction on the mixture with an aqueous solution of ethanol for obtaining extract and a first residue, collecting the extract, wherein the extract is the ethanol extract of the ginseng;

wherein a method for preparing the B) substance comprises: immersing the mint and the orange peel in water, processing distillation extraction for obtaining distillate and a second residue, wherein the distillate is the volatile oil of the mint and the orange peel;

wherein a method for preparing the C) substance comprises: mixing the peony, the poria, the licorice, the peach seed, the yam, the penthorum chienese, the first residue and the second residue, boiling, filtering and collecting filtrate, adding absolute ethanol and waiting for collecting supernatant, wherein the supernatant is the aqueous extract of the ginseng, the salvia, the mint, the orange peel, the peony, the poria, the licorice, the peach seed, the yam and the penthorum chienese;

wherein a method for preparing the D) substance comprises steps of: mixing the fruits and the vegetables, a bacteria liquid of Lactobacillus acidophilus, the bacteria liquid of the Bifidobactreium longum, the bacteria liquid of the Lactobacillus delbrueckii subsp bulgaricus and the bacteria liquid of the Streptococcus thermophilus, fermenting for obtaining a fermentation product, wherein the fermentation product is the fermentation conversion solution of fruits and vegetables.

A mass ratio of the ginseng, the peony, the poria, the licorice, the orange peel, the yam, the mint, the peach seed, the salvia and the penthorum chienese is (1.5˜2.5): (1.5˜2.5): (1.9˜2.9): (0.5˜1.5): (0.8˜1.7): (1.5˜2.5): (0.8˜1.7): (0.8˜1.7): (1.5˜2.5): (3.5˜4.5);

preferably, the mass ratio of the ginseng, the peony, the poria, the licorice, the orange peel, the yam, the mint, the peach seed, the salvia and the penthorum chienese is (1.5, 2 or 2.5): (1.5, 2 or 2.5): (1.9, 2.4 or 2.9): (0.5, 1 or 1.5): (0.8, 1.2 or 1.7): (1.5, 2 or 2.5): (0.8, 1.2 or 1.7): (0.8, 1.2 or 1.7): (1.5, 2 or 2.5): (3.5, 4 or 4.5).

Preferably, in the methods for preparing the A) substance:

reflux extraction time is 2 h˜5 h, the reflux extraction time is preferably 2 h, 3 h or 5 h;

a concentration of the aqueous solution of ethanol is 70%˜95% (according to volume fraction); the concentration is preferably 70%, 90% or 95%;

wherein in the methods for preparing the B) substance:

immersion time is 1 min˜60 min, the immersion time is preferably 1 min, 30 min or 60 min;

a distillation temperature is 90° C.˜100° C., the distillation temperature is preferably 90° C., 95° C. or 100° C.

wherein in the method for preparing the C) substance:

a boiling temperature is 90° C.˜100° C., the boiling temperature is preferably 90° C., 95° C. or 100° C.; a boiling time is 1 h˜2 h, the boiling time is preferably 1 h or 2 h;

a method for adding the absolute ethanol comprises adding the ethanol into the filtrate until a volume percentage of the ethanol in the filtrate is 40%;

fermentation temperature is 18° C.˜37° C., the fermentation temperature is preferably 18° C., 23° C. or 37° C., fermentation time is 10 days to 180 days, the fermentation time is preferably 10 days, 15 days or 180 days, a fermentation method comprises stirring while fermenting.

The method for preparing the A) substance further comprises steps of:

removing the ethanol from the extract, and then concentrating and drying for obtaining a dried product, wherein the dried product is the ethanol extract of the ginseng and the salvia;

the method for preparing the C) substance further comprises steps of:

removing the ethanol from the supernatant after collecting the supernatant, filtering and collecting the filtrate, and then concentrating and drying for obtaining the dried product, wherein the dried product is the aqueous extract of the ginseng, the salvia, the mint, the orange peel, the peony, the poria, the licorice, the peach seed, the yam and the penthorum chienese;

the method for preparing the D) substance further comprises a step of crushing the fruits and vegetables into 40˜50 meshes size before the fermentation;

after the fermentation, the method for preparing the D) substance further comprises steps of: filtering the fermentation product, collecting the filtrate, ultrafiltering, collecting ultrafiltrate, wherein the ultrafiltrate is the fermentation conversion solution of fruits and vegetables.

Preferably, ultrafiltration is filtering the filtrate with 100,000 molecular weight ultrafiltration membrane (an inputting pressure is 1.3 kg, an outputting pressure is 0.5 kg).

The fruits and vegetables are the mixture of the following 54 kinds of fruits and vegetables:

konjac, eggplant, asparagus, spinach, bean sprout, broccoli, cabbage, turnip, cucumber, pea, red pepper, celery, scallion, garlic, grape, grapefruit, watermelon, peach, orange, blueberry, orange, banana, lychee, bitter gourd, leek, pomegranate, dragon fruit, carrot, tomato, Chinese cabbage, celery, bell pepper, lettuce, pear, ginger, taro, bean, pumpkin, lotus root, cherry, kiwi, plum, strawberry, fig, kumquat, citrus, Southland pear, melon, cantaloupe, pawpaw, onion, mulberry, sugar beet and lemon.

Preferably, each fruit or vegetable is of the same weight.

In the method, 1 L the mixture is prepared by: mixing (5˜50) g the ethanol extract of the ginseng and the salvia, (5˜50) g the volatile oil of the mint and the orange peel, (50˜200) g the aqueous extract of the ginseng, the salvia, the mint, the orange peel, the peony, the poria, the licorice, the peach seed, the yam and the penthorum chienese and the fermentation conversion solution of the fruits and vegetables, adding the fermentation conversion solution of fruits and vegetables for making up a volume to 1 L;

preferably, 1 L the mixture is prepared by: mixing (5, 15 or 50) g the ethanol extract of the ginseng and the salvia, (5, 20 or 50) g the volatile oil of the mint and the orange peel, (50, 160 or 200) g the aqueous extract of the ginseng, the salvia, the mint, the orange peel, the peony, the poria, the licorice, the peach seed, the yam and the penthorum chienese and the fermentation conversion solution of the fruits and vegetables, adding the fermentation conversion solution of fruits and vegetables for making up a volume to 1 L;

in the method for preparing the A) substance:

a ratio of the aqueous solution of the ethanol, the ginseng and the salvia is (1˜10) ml: 0.5 g: 0.5 g;

preferably, the ratio of the aqueous solution of the ginseng and the salvia is (1, 5 or 10) ml: 0.5 g: 0.5 g;

in the method for preparing the B) substance:

the ratio of the mint, the orange peel and the water is (1˜10) g: (1˜10) g: (1˜10) ml;

preferably, the ratio of the mint, the orange peel and the water is (1, 5 or 10) g: (1, 5 or 10) g: (1, 5 or 10) ml;

in the method for preparing the C) substance:

the ratio of the peony, the poria, the licorice, the peach seed, the yam, the penthorum chienese and the water is (1˜10) g: (1˜10) g: (1˜10) g: (1˜10) g: (1˜10) g: (1˜10) g: (10˜100) ml;

preferably, the ratio of the peony, the poria, the licorice, the peach seed, the yam, the penthorum chienese and the water is (1, 2 or 10) g: (1, 2.4 or 10) g: (1, 5 or 10) g: (1, 1.2 or 10) g: (1, 2 or 10) g: (1, 4 or 10) g: (50, 60 or 100) ml;

wherein the penthorum chienese is a dried aboveground part of penthorum chinense pursh of the family Saxifragaceae of the genus Penthorum.

In the method for preparing the D) substance:

the ratio of the bacteria liquid of the Lactobacillus acidophilus, the bacteria liquid of the Bifidobactreium longum, the bacteria liquid of the Lactobacillus delbrueckii subsp bulgaricus, the bacteria liquid of the Streptococcus thermophilus, the fermentation conversion solution of the fruits and vegetables and water is (2000˜8000) ml: (2000˜8000) ml: (2000˜8000) ml: (2000˜8000) ml: (1000˜1500) kg: (1000˜1500) kg;

preferably, the ratio of the bacteria liquid of the Lactobacillus acidophilus, the bacteria liquid of the Bifidobactreium longum, the bacteria liquid of the Lactobacillus delbrueckii subsp bulgaricus, the bacteria liquid of the Streptococcus thermophilus, the fermentation conversion solution of the fruits and vegetables and the water is (2000, 5000 or 8000) ml: (2000, 5000 or 8000) ml: (2000, 5000 or 8000) ml: (2000, 5000 or 8000) ml: (1000, 1200 or 1500) kg: (1000, 1200 or 1500) kg.

A method for preparing the bacteria liquid of the Lactobacillus acidophilus comprises: fermenting and growing Lactobacillus acidophilus for obtaining the fermentation production, wherein the fermentation production is the bacteria liquid of the Lactobacillus acidophilus; wherein the fermentation temperature is 20° C.˜41° C., the fermentation temperature is preferably 20° C., 37° C. or 41° C., the fermentation time is 15 h˜36 h, the fermentation time is preferably 15 h, 16 h or 36 h;

a method for preparing the bacteria liquid of the Bifidobactreium longum comprises: fermenting and growing Bifidobactreium longum for obtaining the fermentation production, wherein the fermentation production is the bacteria liquid of the Bifidobactreium longum; wherein the fermentation temperature is 20° C.˜41° C., the fermentation temperature is preferably 20° C., 37° C. or 41° C., the fermentation time is 15 h˜36 h, the fermentation time is preferably 15 h, 16 h or 36 h;

a method for preparing the bacteria liquid of the Lactobacillus delbrueckii subsp bulgaricus comprises: fermenting and growing Lactobacillus delbrueckii subsp bulgaricus for obtaining the fermentation production, wherein the fermentation production is the bacteria liquid of the Lactobacillus delbrueckii subsp bulgaricus; wherein the fermentation temperature is 20° C.˜41° C., the fermentation temperature is preferably 20° C., 37° C. or 41° C., the fermentation time is 15 h˜36 h, the fermentation time is preferably 15 h, 16 h or 36 h;

a method for preparing the bacteria liquid of the Streptococcus thermophilus comprises: fermenting and growing Streptococcus thermophilus for obtaining the fermentation production, wherein the fermentation production is the bacteria liquid of the Streptococcus thermophilus; wherein the fermentation temperature is 20° C.˜41° C., the fermentation temperature is preferably 20° C., 37° C. or 41° C., the fermentation time is 15h-36h, the fermentation time is preferably 15 h, 16 h or 36 h.

The Lactobacillus acidophilus is the Lactobacillus acidophilus CGMCC (China General Microbiological Culture Collection) 1.1854, the Bifidobactreium longum is the Bifidobactreium longum CGMCC 1.2186, the Lactobacillus delbrueckii subsp bulgaricus is the Lactobacillus delbrueckii subsp bulgaricus CGMCC 1.1480, the Streptococcus thermophilus is the Streptococcus thermophilus CGMCC 1.2471.

A growth medium prescription for fermental cultivation: mixing 10 g peptone, 10 g beef extract, 5 g yeast extract, 20 g glucose, 1 g Tween-80, 2 g K₂HPO₄, 1 g Tween-80, 5 g NaAC, 2 g triammonium citrate, 0.2 g MgSO₄, 0.05 g MnSO₄ and the water, making up to 1 L by the water for obtaining the growth medium. The pharmaceutical composition prepared by the above methods for treating the melasma is also within protection scope of the present invention.

The pharmaceutical composition for treating the melasma prepared by the method is within the scope of the present invention.

Experiments of the present invention illustrates that extracting time for obtaining the ethanol extract of the ginseng and the salvia, the volatile oil of the mint and the orange peel, the aqueous extract of the ginseng, the salvia, the mint, the orange peel, the peony, the poria, the licorice, the peach seed, the yam and the penthorum chienese and the fermentation conversion solution of the fruits and vegetables is short.

These and other objectives, features, and advantages of the present invention will become apparent from the following detailed description, the accompanying drawings, and the appended claims.

### DETAILED DESCRIPTION OF THE PREFERRED EMBODIMENT

Experimental methods used in the following preferred embodiments are conventional methods except for special instructions.

Ingredients and agents used in the following preferred embodiments are commercially available except for special instructions.

Preferred embodiment 1: obtaining ferment composition of ginseng for treating melasma

Method I:

wherein the active ingredients of the ferment composition of the ginseng are: 200 g the ginseng, 200 g peony, 240 g poria, 100 g licorice, 120 g orange peel, 200 g yam, 120 g mint, 120 g peach seed, 200 g salvia and 400 g penthorum chienese;

a) preparing ethanol extract of the ginseng and the salvia:

1. preparing the ingredient: wherein the ginseng and the salvia purchased from Jilin Zixin Pharmaceutical Industrial Co., Ltd are in line with the Pharmacopoeia of the People's Republic of China; quality is strictly controlled before feeding according to corporate standards;

2. weighting the ginseng and the salvia according to a prescription, washing with water and drying the ginseng and the salvia, processing reflux extraction twice with a 10-flod (v/w) 90% aqueous solution of ethanol, reflux extraction time for once is 3 h, filtrating extract, combining filtrate, vacuum recovering the ethanol; wherein residues are ginseng residues and salvia residues; and

3. concentrating the filtrate to a relative density of 1.10˜1.15 (60° C., 0.07 MPa), spray-drying the filtrate (an inputting temperature is 170° C., an outputting temperature is 70° C.) for obtaining spray-dried powder, wherein the powder is the ethanol extract of the ginseng and the salvia;

b) preparing volatile oil of the mint and the orange peel:

1. preparing the ingredient: wherein the mint and the orange peel purchased from Jilin Zixin Pharmaceutical Industrial Co., Ltd are in line with the Pharmacopoeia of the People's Republic of China; the quality is strictly controlled before feeding according to the corporate standards; and

2. weighting the mint and the orange peel according to the prescription, washing with the water and drying the mint and the orange peel, wherein immersion time is 30 min, the water should fully immerse the mint and the orange peel; adding 5-flod the water, distilling at 100° C., collecting distillate with another container with a ratio of 5:1 (wherein a volume of the distillate is ⅕ of the volume of the water for completely collecting the distillate), wherein the residues are mint residues and orange peel residues; the ration of the mint, the orange peel and the water is 5 g: 5 g: 5 ml;

c) preparing aqueous extract of the ginseng, the salvia, the mint, the orange peel, the peony, the poria, the licorice, the peach seed, the yam and the penthorum chienese:

1. preparing the ingredient: wherein the peony, the poria, the licorice, the peach seed, the yam and the penthorum chienese purchased from Jilin Zixin Pharmaceutical Industrial Co., Ltd are in line with the Pharmacopoeia of the People's Republic of China; the quality is strictly controlled before feeding according to the corporate standards;

2. weighting the peony, the poria, the licorice, the peach seed, the yam and the penthorum chienese according to the prescription, washing with the water and drying the peony, the poria, the licorice, the peach seed, the yam and the penthorum chienese; and

3. mixing the ginseng residues, the salvia residues, the mint residues, the orange peel residues, the peony, the poria, the licorice, the peach seed, the yam and the penthorum chienese, boiling at 100° C. twice, wherein the water is respectively 6 times and 5 times of the mixture, boiling time is respectively 2 h and 1 h; combining twice and concentrating the mixture until a relative density is 1.12˜1.20 (by vacuum concentrating), adding ethanol and stirring until a volume percentage of the ethanol is 40%, waiting for 24 h, collecting supernatant and filtering after the ethanol is completely recovered; concentrating the filtrate to the relative density of 1.10˜1.15 (60° C., 0.07 MPa), spray-drying the filtrate (the inputting temperature is 170° C., the outputting temperature is 70° C.) for obtaining the spray-dried powder, wherein the powder is the aqueous extract of the ginseng, the salvia, the mint, the orange peel, the peony, the poria, the licorice, the peach seed, the yam and the penthorum chienese, the ratio of the peony, the poria, the licorice, the peach seed, the yam, the penthorum chienese and the water is 2 g: 2.4 g: 1 g: 1.2 g: 2 g: 4 g: 60 ml;

d) preparing a fermentation conversion solution of fruits and vegetables:

1. selecting the fruits and vegetables materials;

TABLE 1
fruits and vegetables selected

| Ingredient | nutritional content |
|---|---|
| konjac | vitamin Bl, B2, citric acid, |
| | fermentation conversion matter |
| eggplant | vitamin A, Bl, B2, C, |
| | fermentation conversion matter |
| asparagus | vitamin Bl, B2, citric acid, |
| | fermentation conversion matter |
| spinach | vitamin A, C, calcium, iron, |
| | fermentation conversion matter |
| bean sprout | vitamin, saponin, amino acid, |
| | fermentation conversion matter |
| broccoli | vitamin Bl, B2, citric acid, |
| | fermentation conversion matter |
| cabbage | vitamin Bl, B2, citric acid, |
| | fermentation conversion matter |
| turnip | vitamin Bl, B2, citric acid, |
| | fermentation conversion matter |
| cucumber | vitamin Bl, B2, citric acid, |
| | fermentation conversion matter |
| pea | vitamin Bl, B2, citric acid, |
| | fermentation conversion matter |
| red pepper | vitamin Bl, B2, citric acid, |
| | fermentation conversion matter |
| celery | vitamin Bl, B2, citric acid, |
| | fermentation conversion matter |
| scallion | vitamin Bl, B2, citric acid, |
| | fermentation conversion matter |
| garlic | vitamin Bl, B2, citric acid, |
| | fermentation conversion matter |
| grape | vitamin Bl, B2, citric acid, |
| | fermentation conversion matter |
| grapefruit | vitamin Bl, B2, citric acid, |
| | fermentation conversion matter |
| watermelon | vitamin Bl, B2, citric acid, |
| | fermentation conversion matter |
| peach | vitamin Bl, B2, citric acid, |
| | fermentation conversion matter |
| orange | vitamin Bl, B2, C, |
| | fermentation conversion matter |
| blueberry | vitamin Bl, B2, citric acid, |
| | fermentation conversion matter |
| orange | vitamin Bl, B2, citric acid, |
| | fermentation conversion matter |
| banana | vitamin Bl, B2, citric acid, |
| | fermentation conversion matter |
| lychee | vitamin Bl, B2, citric acid, |
| | fermentation conversion matter |
| bitter gourd | vitamin Bl, B2, citric acid, |
| | fermentation conversion matter |
| leek | vitamin Bl, B2, citric acid, |
| | fermentation conversion matter |
| pomegranate | vitamin Bl, B2, citric acid, |
| | fermentation conversion matter |
| dragon fruit | vitamin Bl, B2, citric acid, |
| | fermentation conversion matter |
| carrot | vitamin A, carotene, |
| | fermentation conversion matter |
| tomato | vitamin A, carotene, citric acid, |
| | fermentation conversion matter |
| Chinese cabbage | vitamin, mineral substance, |
| | fermentation conversion matter |
| celery | vitamin, mineral substance, cellulose, |
| | fermentation conversion matter |
| bell pepper | vitamin C, mineral substance, |
| | fermentation conversion matter |
| lettuce | vitamin A, mineral substance, |
| | fermentation conversion matter |
| pear | fructose, mineral substance, |
| | fermentation conversion matter |
| ginger | vitamin, mineral substance, |
| | fermentation conversion matter |
| taro | vitamin Bl, B2, C, mineral substance, |
| | fermentation conversion matter |
| bean | vitamin Bl, B2, citric acid, carotene, |
| | fermentation conversion matter |
| pumpkin | carotene, mineral substance, |
| | fermentation conversion matter |
| lotus root | iron, tannin, fermentation conversion matter |
| cherry | mineral substance, |
| | fermentation conversion matter |
| kiwi | vitamin C, fermentation conversion matter |
| plum | organic acid, vitamin, |
| | fermentation conversion matter |
| strawberry | vitamin C, mineral substance, ellagic acid, |
| | fermentation conversion matter |
| fig | digest fermentation conversion matter, |
| | vitamin, mineral substance |
| kumquat | vitamin Bl, B2, C, |
| | fermentation conversion matter |
| citrus | vitamin C, citric acid, |
| | fermentation conversion matter |
| Sounthland pear | vitamin Bl, B2, C, citric acid, |
| | fermentation conversion matter |
| melon | vitamin Bl, B2, citric acid, |
| | fermentation conversion matter |
| cantaloupe | fructose, potassium, vitamin A, |
| | fermentation conversion matter |
| pawpaw | vitamin B, C, E, citric acid, carotene, |
| | fermentation conversion matter |
| onion | vitamin B, C, carotene, |
| | fermentation conversion matter |
| mulberry | vitamin Bl, B2, citric acid, |
| | fermentation conversion matter |
| sugar beet | betaine, fermentation conversion matter |
| lemon | citric acid, fermentation conversion matter |

2. fermenting and converting the fruits and vegetables, comprising steps of:

2-a) selecting and purchasing cultures;

wherein probiotics culture is purchased from Institute of Microbiology Chinese Academy of Sciences, probiotics is respectively Lactobacillus acidophilus CGMCC 1.1854, Bifidobactreium longum CGMCC 1.2186, Lactobacillus delbrueckii subsp bulgaricus CGMCC 1.1480 and Streptococcus thermophilus CGMCC 1.2471, the end of all the cultures are preserved as a starter community in sand tubes;

2-b) preparing main seeds;

wherein preparation of the probiotic main seed comparses steps of (the main seed should be reproduced for no more than ten generations, and the main seed in the preparation is reproduced for four generations):

2-b1) respectively taking a 1/10 sand tube amount of the Lactobacillus acidophilus CGMCC 1.1854, the Bifidobactreium longum CGMCC 1.2186, the Lactobacillus delbrueckii subsp bulgaricus CGMCC 1.1480 and the Streptococcus thermophilus CGMCC 1.2471 with a sterile stainless steel spoon, freezing and storing the rest, respectively connecting the sand tubes to 50 ml (250 flask) MRS liquid growth medium (the growth medium comprises 10 g/l peptone, 10 g/l beef extract, 5 g/l yeast extract, 20 g/l glucose, 1 g/l Tween-80, 2 g/l K₂HPO₄, 1 g/l Tween-80, 5 g/l NaAC, 2 g/l triammonium citrate, 0.2 g/l MgSO₄ and 0.05 g/l MnSO₄, the growth medium is sterilized at 121° C. for 20 min), growing at 37° C. with a 100 rpm shaker for 16 h;

2-b2) picking a loop of each colony, respectively streaking the colony loop on MRS solid growth medium (the growth medium comprises 10 g/l peptone, 10 g/l beef extract, 5 g/l yeast extract, 20 g/l glucose, 1 g/l Tween-80, 2 g/l K₂HPO₄, 1 g/l Tween-80, 5 g/l NaAC, 2 g/l triammonium citrate, 0.2 g/l MgSO₄, 0.05 g/l MnSO₄ and 1.5% agar, the growth medium is sterilized at 121° C. for 20 min), growing at 37° C. with an incubator for 16 h;

2-b3) picking the best-growing colony form each the loop, respectively connecting the colony to the 50 ml MRS liquid growth medium, growing at 30° C. with the 180 rpm shaker for 16 h; and

2-b4) respectively connecting to the 500 ml MRS liquid growth medium, growing at 37° C. with the 100 rpm shaker for 16 h, adding glycerol until the glycerol takes a proportion of 20%, shaking up, respectively dividing into 1 ml and inputting into a freezing tube as the main seed of the probiotic Lactobacillus acidophilus, the Bifidobactreium longum, the Lactobacillus delbrueckii subsp bulgaricus or the Streptococcus thermophilus, preservating at −40° C.;

3. preparing working seeds:

wherein preparation of probiotic working seed comparses steps of (the working seed should be reproduced for no more than five generations, and the working seed in the preparation is reproduced for four generations):

3-a) respectively taking the main seeds of the Lactobacillus acidophilus CGMCC 1.1854, the Bifidobactreium longum CGMCC 1.2186, the Lactobacillus delbrueckii subsp bulgaricus CGMCC 1.1480 and the Streptococcus thermophilus CGMCC 1.2471 with a sterile inoculating loop, respectively streaking to the MRS solid growth medium, growing at 37° C. with the incubator for 16 h;

3-b) picking the best-growing colony form each the loop, respectively connecting the colony to the 50 ml MRS liquid growth medium, growing at 37° C. with the 100 rpm shaker for 16 h;

3-c) respectively connecting to the 500 ml MRS liquid growth medium, growing at 37° C. with the 100 rpm shaker for 16 h; and 3-d) respectively connecting to the 5000 ml MRS liquid growth medium, growing at 37° C. with the 100 rpm shaker for 16 h, obtaining the bacteria liquid of the probiotic Lactobacillus acidophilus, the Bifidobactreium longum, the Lactobacillus delbrueckii subsp bulgaricus and the Streptococcus thermophilus; wherein the above bacteria liquid is all fermentation products in a fermentation container;

4. processing the fruits and vegetables:

4-a) weighing the fruits and vegetables;

4-b) washing, draining and weighing; and

4-c) crushing into 40˜50 meshes size, putting into a fermentation tank;

wherein for the 3-ton fermentation tank (an effective volume=3 tons×0.8=2.4 tons), an actual feeding amount is controlled at 2400 kg; the fruits and vegetables: water is 1:1, feeding 1200 kg the fruits and vegetables, then adding 1200 kg the water;

5. fermenting and converting a rapid enzymic hydrolysate of the fruits and vegetables:

5-a) respectively adding 5000 ml the bacteria liquid of the Lactobacillus acidophilus CGMCC 1.1854, the Bifidobactreium longum CGMCC 1.2186, the Lactobacillus delbrueckii subsp bulgaricus CGMCC 1.1480 and the Streptococcus thermophilus CGMCC 1.2471, controlling a fermentation temperature at 23° C., stirring for 15 days;

5-b) filtering with 200 meshes size a filter cloth, abandoning pomace, obtaining the filtrate; and

5-c) ultrafiltrating with 100,000 molecular weight ultrafiltration membrane (an inputting pressure is 1.3 kg, an outputting pressure is 0.5 kg), obtaining clear liquid, sealing and preserving at 4° C., wherein the clear liquid is the fermentation conversion solution of the fruits and vegetables; and

6. testing:

wherein the main products of the fermentation conversion solution of the fruits and vegetables is mainly lactic acid and acetic acid, therefore, an acidity is identified as a characteristic component;

the acidity of the fermentation conversion solution of the fruits and vegetables: referring to an amount of a 0.1N NaOH solution used in milliliter for titrating 100 ml the fermentation conversion solution of fruits and vegetables, wherein 10 ml samples are often used in a test;

a method for testing comprises steps of: taking 10 ml the fermentation conversion solution of the fruits and vegetables, adding 20 ml the water and 0.5 ml 0.5% phenolphthalein indicator, titrating with the 0.1N NaOH until the liquid is reddish and doesn't fade within 30 sec; calculating: the acidity=the amount of the 0.1N NaOH solution used×10;

result: the acidity of the fermentation conversion solution of the fruits and vegetables is 42; and

e) preparing composition:

mixing the above extract and the fermentation conversion solution of the fruits and vegetables according to the following formulation:

mixing 15 g the ethanol extract of the ginseng and the salvia, 20 g the volatile oil of the mint and the orange peel, 160 g the aqueous extract of the ginseng, the salvia, the mint, the orange peel, the peony, the poria, the licorice, the peach seed, the yam and the penthorum chienese and the fermentation conversion solution of the fruits and vegetables, adding the fermentation conversion solution of the fruits and vegetables for making up the volume to 10,000 ml, wherein the mixture is the composition, dividing into 1000 bottles with 10 ml/bottle for future usage.

Method II:

wherein the active ingredients of the ferment composition of ginseng are: 150 g the ginseng, 150 g the peony, 190 g the poria, 50 g the licorice, 80 g the orange peel, 150 g the yam, 80 g the mint, 80 g the peach seed, 150 g the salvia and 350 g the penthorum chienese;

a) preparing the ethanol extract of the ginseng:

the extracting method is basically the same as the method in the method I, deference is that the ginseng is reflux-extracted by the 1-flod (v/w) 70% aqueous solution of the ethanol and the reflux extraction time is 2 h for once;

b) preparing the volatile oil of the mint and the orange peel:

the extracting method is basically the same as the method in the method I, the deference is that the ratio of the mint, the orange peel and the water is 1 g:1 g:1 ml; the immersion time is 1 min; the distillation temperature is 90° C.;

c) preparing aqueous extract of the ginseng, the salvia, the mint, the orange peel, the peony, the poria, the licorice, the peach seed, the yam and the penthorum chienese:

the extracting method is basically the same as the method in the method I, the deference is that the ratio of the peony, the poria, the licorice, the peach seed, the yam, the penthorum chienese and the water is 1 g:1 g:5 g:1 g:1 g:1 g: 50 ml; the boiling time is 90° C.;

d) preparing the fermentation conversion solution of the fruits and vegetables:

the extracting method is basically the same as the method in the method I, the deference is respectively adding 2000 ml the bacteria liquid of the Lactobacillus acidophilus CGMCC 1.1854, the Bifidobactreium longum CGMCC 1.2186, the Lactobacillus delbrueckii subsp bulgaricus CGMCC 1.1480 and the Streptococcus thermophilus CGMCC 1.2471, adding 1000 kg the fruits and vegetables and adding 1000 kg the water;

wherein the fermentation time is 18° C., the fermentation time is 10 days;

wherein in the preparation of the bacteria liquid of the Lactobacillus acidophilus, the Bifidobactreium longum, the Lactobacillus delbrueckii subsp bulgaricus and the Streptococcus thermophilus, the fermentation time is 20° C., the fermentation time is 15 h;

wherein the testing method is the same as the method in the method I, the result has no significant difference; and

e) preparing the composition:

the preparing method is basically the same as the method in the method I, the deference is mixing 5 g the ethanol extract of the ginseng, 5 g volatile oil of the mint and the orange peel, 50 g aqueous extract of the ginseng, the salvia, the mint, the orange peel, the peony, the poria, the licorice, the peach seed, the yam and the penthorum chienese and the fermentation conversion solution of the fruits and vegetables; adding the fermentation conversion solution of the fruits and vegetables for making up the volume to 10,000 ml, wherein the mixture is the composition.

Method III,

wherein the active ingredients of the ferment composition of ginseng are: 250 g the ginseng, 250 g the peony, 290 g the poria, 150 g the licorice, 170 g the orange peel, 250 g the yam, 170 g the mint, 170 g the peach seed, 250 g the salvia and 450 g the penthorum chienese;

a) preparing the ethanol extract of the ginseng:

the extracting method is basically the same as the method in the method I, deference is that the ginseng is reflux-extracted by the 5-flod (v/w) 95% aqueous solution of the ethanol;

b) preparing the volatile oil of the mint and the orange peel:

the extracting method is basically the same as the method in the method I, the deference is that the ratio of the mint, the orange peel and the water is 10 g:10 g:10 ml; the immersion time is 60 min; the distillation temperature is 95° C.;

c) preparing aqueous extract of the ginseng, the salvia, the mint, the orange peel, the peony, the poria, the licorice, the peach seed, the yam and the penthorum chienese:

the extracting method is basically the same as the method in the method I, the deference is that the ratio of the peony, the poria, the licorice, the peach seed, the yam, the penthorum chienese and the water is 10 g:10 g:10 g:10 g:10 g:10 g:100 ml; the boiling time is 95° C.;

d) preparing the fermentation conversion solution of the fruits and vegetables:

the extracting method is basically the same as the method in the method I, the deference is respectively adding 8000 ml the bacteria liquid of the Lactobacillus acidophilus CGMCC 1.1854, the Bifidobactreium longum CGMCC 1.2186, the Lactobacillus delbrueckii subsp bulgaricus CGMCC 1.1480 and the Streptococcus thermophilus CGMCC 1.2471, adding 1500 kg the fruits and vegetables and adding 1500 kg the water;

wherein the fermentation time is 37° C., the fermentation time is 180 days;

wherein in the preparation of the bacteria liquid of the Lactobacillus acidophilus, the Bifidobactreium longum, the Lactobacillus delbrueckii subsp bulgaricus and the Streptococcus thermophilus, the fermentation time is 41° C., the fermentation time is 36 h;

wherein the testing method is the same as the method in the method I, the result has no significant difference; and

i) preparing the composition:

the preparing method is basically the same as the method in the method I, the deference is mixing 50 g the ethanol extract of the ginseng, 50 g volatile oil of the mint and the orange peel, 200 g aqueous extract of the ginseng, the salvia, the mint, the orange peel, the peony, the poria, the licorice, the peach seed, the yam and the penthorum chienese and the fermentation conversion solution of the fruits and vegetables; adding the fermentation conversion solution of the fruits and vegetables for making up the volume to 10,000 ml, wherein the mixture is the composition.

### Preferred Embodiment 2

### Preparing Ginseng Oral Liquid for Treating the Melasma

Conventional production processes of oral liquid are utilized in the present invention: mixing→filtering→instantaneously sterilizing→canning→capping→leak testing→clarity checking→packaging→testing→storing, wherein the processes before the leak testing are provided in 100,000 class clean area. A production process design and equipment conditions are in line with GMP (Good Manufacturing Practice) requirements of China.

Specifically, a production method comprises steps of:

1. mixing the composition in the method I of the preferred embodiment 1 with a sweetener (specifically, the sweetener is white sugar purchased from Changchun Eurasia department store, w/v), then putting into a make-up tank, mixing for 30 min for shaking up, instantaneously sterilizing by high heat, wherein the product is the ginseng oral liquid for treating the melasma;

2. canning the oral liquid: wherein the oral liquid is canned 10 ml per bottle by filling machines, volumes of the canned oral liquid should be detected during canning in such a manner that difference between the volumes is controlled within the range;

3. capping: wherein the bottle is capped by capping machines, the unqualified products are screened out;

4. leak testing: wherein the capped bottles are tested by a vacuum leak detector;

5. inner packaging: wherein cardboard boxes are utilized, and each the box contains 10 bottles;

6. outer packaging: wherein corrugated boxes are utilized;

7. testing: wherein the products are tested according to the corporate standards;

8. storing: wherein the products are stored in warehouses after passing the tests.

### Preferred Embodiment 3

### Study on Anti-Melasma Effects of the Ginseng Oral Liquid for Treating the Melasma

1. Experiment Materials and Methods:

1.1. Testing Medicine:

the ginseng oral liquid for treating the melasma is prepared in the preferred embodiment 2, vitamin C pills are purchased from Sichuan Furui Pharmaceutical Industrial Co., Ltd;

1.2. Animals:

Kunming female mice weighting 28±2 g, provided by the Experimental Animal Center of Jilin University;

1.3. Reagents:

progesterone injection: wherein the progesterone injection is 10 mg/ml and a serial number is 060314, the progesterone injection is purchased from Zhejiang Xianju Pharmaceutical Industrial Co., Ltd; superoxide dismutase (SOD): wherein the serial number is 20060803; malondialdehyde (MDA): wherein the serial number is 20060802; nitrogen monoxide (NO): wherein the serial number is 20060728; coomassie brilliant blue: wherein the serial number is 20060731; reagent kits are all provided by Nanjing Jiancheng Bioengineering Institute; a mouse anti-human McAb (monoclonal antibody) HMB45 (melanoma) ready kit MAB-0098 is produced by Fujian Maixin Pharmaceutical Industrial Co., Ltd; a concentration DAB (diaminobenzidine) kit is produced by Beijing Zhongshan Jinqiao Pharmaceutical Industrial Co., Ltd; and

1.4. The Experimental Methods:

randomly dividing 84 the mice into six groups; injecting the mice of a normal group with sterilized water with a dosage of 2 ml/kg; injecting the mice of the other groups with the progesterone in muscles of rear legs, wherein the left rear leg and the right rear leg are injected alternatively, the dosage is 20 mg/kg (equals to eight times of a clinical dosage of the human); injecting for 6 d a week and lasting for 30 d; feeding the medicine or distilled water with the dosage of 0.2 ml/10 g while modeling, wherein the medicine or the distilled water are fed once a day for 30 d; wherein the dosages of a low group, a medium group and a high group are 12, 24 and 36 g/kg of the ginseng oral liquid (equals to five, ten, fifteen times of the clinical dosage); the dosage of the vitamin C is 0.1 g/kg (equals to ten times of a clinical dosage of the human); sampling 1 ml blood from orbits of the mice of each the group 1 h after the last feeding, putting in a capped plastic tube comprising dried heparin, detecting a viscosity the whole blood; executing all the mice and removing hairs on backs, collecting livers and skins, washing with precooled saline, drying with filter paper; taking 0.5 g skin and 0.5 g liver, adding into the cold saline with the ratio of 1:9 (w/v), processing bath for obtaining 10% tissue homogenate, separating by a 2500 r/min centrifugal separator, taking the supernatant and detecting relative parameters;

1.4.1. Effects on the Viscosity of the Whole Blood of the Mice:

detecting the viscosity of the whole blood by a blood viscosity analyzer, wherein it is illustrated by table 2 that when compared with the normal control group, the blood viscosities of the mice of the modeling control group and the vitamin C group with different shear rates are obviously increased; when compared with the modeling control group, the blood viscosities of the high group with the ginseng oral liquid for treating the melasma are obviously decreased, which means that the ferment composition of the ginseng can effectively improve a rheology of the blood of the mouse with the melasma as well as decrease the blood viscosity;

TABLE 2
effects of ferment composition of ginseng on viscosity of whole blood of mice (x ± s)

| | dosage | num- | blood viscosity with different shear rates (mPas) | | | |
|---|---|---|---|---|---|---|
| group | (ml) | ber | 1/s | 3/3 | 30/s | 100/s |
| normal | 0 | 10 | 22.74 ± 3.08 | 16.58 ± 2.48 | 8.20 ± 1.90 | 3.66 ± 0.84* |
| control | | | | | | |
| modeling | 0 | 11 | 30.93 ± 5.70 | 21.81 ± 4.38 | 11.39 ± 2.80 | 4.83 ± 1.73 |
| control | | | | | | |
| high | 30 | 9 | 22.26 ± 3.12* | 18.13 ± 2.85* | 9.33 ± 2.30* | 3.85 ± 1.03 |
| medium | 20 | 10 | 24.12 ± 4.26 | 19.11 ± 3.34 | 10.00 ± 2.33 | 4.00 ± 1.17 |
| low | 10 | 9 | 27.84 ± 2.76 | 18.96 ± 4.04 | 10.24 ± 2.08 | 4.27 ± 0.78 |
| vitamin C | 0.1 | 10 | 29.22 ± 2.18 | 28.85 ± 4.35 | 11.22 ± 1.75 | 4.60 ± 1.36 |

Note:
comparison with the modeling groups,
*P < 0.05,
**P < 0.01 (same as below);

1.4.2. Detecting SOD, MDA and NO Parameters:

detecting with a 722 s spectrophotometer according to instructions of the reagent kits and operation requirements; wherein effects on the SOD, the MDA and the NO contents in mice livers are illustrated in table 3, when compared with the normal control group, SOD activities in the livers of the mice of the modeling control group are decreased, MDA and NO contents are increased, and great difference exists in the MDA content; when compared with the modeling control group, the SOD activities of the high group with the ginseng oral liquid and the vitamin C group are obviously increased, and the MDA contents of the high group are obviously decreased;

TABLE 3
effects of ferment composition of ginseng on SOD,
MDA and NO contents in mice livers (x ± s)

| | dosage | num- | | | |
|---|---|---|---|---|---|
| group | (ml) | ber | SOD | MDA | NO |
| normal | 0 | 10 | 37.47 ± 18.05 | 3.25 ± 1.61 | 1.24 ± 0.94 |
| control | | | | | |
| modeling | 0 | 11 | 32.48 ± 8.62 | 5.53 ± 2.55 | 1.98 ± 1.17 |
| control | | | | | |
| high | 30 | 9 | 52.15 ± 14.82* | 3.78 ± 1.51* | 1.66 ± 0.60 |
| medium | 20 | 10 | 42.76 ± 16.20 | 4.5 ± 2.04 | 1.60 ± 1.00 |
| low | 10 | 9 | 36.15 ± 10.77 | 4.62 ± 1.21 | 1.98 ± 0.83 |
| vitamin C | 0.1 | 10 | 45.62 ± 16.04* | 4.58 ± 1.26 | 1.90 ± 0.93 |

wherein the effects on the SOD, the MDA and the NO contents in mice skins are illustrated in table 4, when compared with the normal control group, the SOD activities in the skins of the mice of the modeling control group are decreased, the MDA and the NO contents are increased, and great difference exists in the MDA content; when compared with the modeling control group, the SOD activities of the high group with the ginseng oral liquid and the medium group are obviously increased, and the MDA contents of the high group are obviously decreased; when compared with the modeling control group, the MDA contents of the vitamin group are obviously decreased;

TABLE 4
effects of ferment composition of ginseng on
SOD, MDA and NO contents in mice skins (x ± s)

| | dosage | num- | | | |
|---|---|---|---|---|---|
| group | (ml) | ber | SOD | MDA | NO |
| normal | 0 | 10 | 15.94 ± 3.73 | 3.50 ± 1.08 | 1.17 ± 0.67 |
| control | | | | | |
| modeling | 0 | 10 | 14.85 ± 5.07 | 5.40 ± 1.06 | 1.73 ± 0.79 |
| control | | | | | |
| high | 30 | 10 | 23.91 ± 9.17* | 3.54 ± 1.24* | 1.25 ± 0.45 |
| medium | 20 | 10 | 21.80 ± 6.24* | 3.83 ± 1.21* | 1.72 ± 0.59 |
| low | 10 | 10 | 18.87 ± 6.30 | 4.58 ± 1.13 | 1.72 ± 0.92 |
| vitamin C | 0.1 | 10 | 19.95 ± 5.33 | 4.20 ± 1.34* | 1.71 ± 0.52 |

1.4.3. Observing Melanin Cells in the Skins According to Pathomorphology:

Stripping a block of the back skin of the mouse, removing the hairs, fixing with 10% neutral formalin, embedding in conventional paraffin, cutting into 5 μm thick sections continuously, and staining with HE (hematoxylin-eosinstaining); marking the melanin by immunohistochemistry method, utilizing biotin method for obtaining streptomyces, selecting the mouse anti-human McAb HMB45 (melanoma) as a first anti-body, coloring by the DAB, and coloring again with hematoxylin; processing negative control with PBS (phosphate buffered saline) instead of the first anti-body; processing computer image analysis on melanin-positive target: randomly selecting skins form eight mice of each group, obtaining a tissue section from each the skin, observing five non-overlapping fields of view, and finding the positive targets; quantitatively analyzing by a MIAS2000 computer image analysis system for obtaining 5 views of each the tissue section and average size, average optical density and integrated optical density of the melanin-positive target of the eight animals of each the group;

wherein observing the melanin cells stained by the HE in the skins according to the pathomorphology: the melanin cells can not be found under a light microscope; observing the HMB45 mark: a small number of the melanin cells can be found in hair follicles and sebaceous gland basal cells of the mice of the normal control group, the melanin cells cluster in the hair follicles and the sebaceous gland basal cells of the mice of the modeling control group, and can also be found in epidermis basal cells and prickle cell layer, the melanin cells only scatter in the basal cells of basal layer and the hair follicles of the mice of the four groups with the medicine;

effects on the melanin cells in the mice skins are illustrated in table 5, wherein when compared with the normal control group, the average size, the average optical density and the integrated optical density of the mouse of the modeling control group are significantly increased; when compared with the modeling control group, the average optical density and the integrated optical density of the high and medium groups are significantly decreased;

TABLE 5
effects of ferment composition of ginseng on melanin cells in skins of mice (x ± s)

| | | | average size of | average optical | integrated optical |
|---|---|---|---|---|---|
| group | dosage (ml) | number | positive target | density | density |
| normal | 0 | 10 | 2893.47 ± 923.51 | 0.24 ± 0.08 | 851.52 ± 314.35 |
| control | | | | | |
| modeling | 0 | 11 | 4116.55 ± 1175.89 | 0.39 ± 0.11 | 1421.52 ± 532.47 |
| control | | | | | |
| high | 30 | 9 | 3480.95 ± 1206.12 | 0.26 ± 0.09* | 883.15 ± 345.00* |
| medium | 20 | 10 | 3620.40 ± 1204.21 | 0.28 ± 0.10* | 985.27 ± 321.91* |
| low | 10 | 9 | 3910.85 ± 1365.11 | 0.34 ± 0.12 | 1226.52 ± 453.44 |
| vitamin C | 0.1 | 10 | 3720.56 ± 1191.13 | 0.33 ± 0.14 | 1152.52 ± 344.09 |

The melasma is a multi-factor, multi-system, multi-link pigmented skin problem, pathogenesis thereof is complex, and a real cause is not yet clear. As a pigmented skin problem, too much melanin generated is a basic reason, synthesis of the melanin by the melanin cells is a very complex process, and is affected by a variety of factors. Hemodynamic parameters of patients with the melasma are abnormal, therefore, changes of the hemodynamic parameters are considered to have a close relationship with the melasma.

Detection of the hemodynamic parameters provides an experimental basis for treating the disease.

In recent years, many studies have indicated that oxygen free radicals are responsible for formation of the melanin and pigmentation in the skin. The MDA can lead to cross-link of protein molecules for forming a fluorescent chromophore (pigment). In addition to the effect of removing the free radicals, the SOD can also inhibit the activity of tyrosinase and reduce the pigmentation. A large amount of data indicated that the activities of the SOD and catalase (CAT) in the blood of the patient with the melasma are obviously decreased, and the contents of LP0 and the MDA are obviously increased. The above studies illustrate that imbalance between oxidation and antioxidant may be an important factor for the melasma.

In many signal-medicated methods for affecting function of the melanin cells, the NO is considered to be an extremely important signal molecule with an important role. The NO can stimulate the activity of the tyrosinase, and improve the amount of the new synthesized melanin.

Experiment results according to the present invention indicated that the ginseng oral liquid for treating the melasma has the effects of promoting blood circulation and removing blood stasis, and can significantly reduce the viscosity of the whole blood of the mice with the melasma and improve the viscosity of the blood; in addition, the activities of the SOD in the livers and skins of the mice of the modeling group with the melasma is obviously decreased when compared with the normal group, and the contents of the MDA and the NO are higher; and after feeding the pharmaceutical composition, the content of the MDA is obviously decreased and the activity of the SOD is increased, which illustrates that ginseng oral liquid for treating the melasma may reduce the formation of the melanin by decreasing the activity of the free radical scavenging enzyme and reducing lipid peroxidation.

One skilled in the art will understand that the embodiment of the present invention as shown in the drawings and described above is exemplary only and not intended to be limiting.

It will thus be seen that the objects of the present invention have been fully and effectively accomplished. Its embodiments have been shown and described for the purposes of illustrating the functional and structural principles of the present invention and is subject to change without departure from such principles. Therefore, this invention includes all modifications encompassed within the spirit and scope of the following claims.

1-9. (canceled)

10. A method for preparing a pharmaceutical composition for treating melasma, comprising:
mixing the following four substances for obtaining a mixture, wherein the mixture is the pharmaceutical composition for treating the melasma, wherein the four substances comprises:
A) ethanol extract of ginseng and salvia; B) volatile oil of mint and orange peel; C) aqueous extract of the ginseng, the salvia, the mint, the orange peel, peony, poria, licorice, peach seed, yam and penthorum chienese; D) a fermentation conversion solution of fruits and vegetables;
wherein a method for preparing the A) substance comprises: mixing the ginseng and the salvia, processing reflux extraction on the mixture with an aqueous solution of ethanol for obtaining extract and a first residue, collecting the extract, wherein the extract is the ethanol extract of the ginseng;
wherein a method for preparing the B) substance comprises: immersing the mint and the orange peel in water, processing distillation extraction for obtaining distillate and a second residue, wherein the distillate is the volatile oil of the mint and the orange peel;
wherein a method for preparing the C) substance comprises: mixing the peony, the poria, the licorice, the peach seed, the yam, the penthorum chienese, the first residue and the second residue, boiling, filtering and collecting filtrate, adding absolute ethanol and waiting for collecting supernatant, wherein the supernatant is the aqueous extract of the ginseng, the salvia, the mint, the orange peel, the peony, the poria, the licorice, the peach seed, the yam and the penthorum chienese;
wherein a method for preparing the D) substance comprises steps of: mixing the fruits and the vegetables, a bacteria liquid of Lactobacillus acidophilus, the bacteria liquid of the Bifidobactreium longum, the bacteria liquid of the Lactobacillus delbrueckii subsp bulgaricus and the bacteria liquid of the Streptococcus thermophilus, fermenting for obtaining a fermentation product, wherein the fermentation product is the fermentation conversion solution of fruits and vegetables.

11. The method, as recited in claim 10, wherein:
a mass ratio of the ginseng, the peony, the poria, the licorice, the orange peel, the yam, the mint, the peach seed, the salvia and the penthorum chienese is (1.5˜2.5): (1.5˜2.5): (1.9˜2.9): (0.5˜1.5): (0.8˜1.7): (1.5˜2.5): (0.8˜1.7): (0.8˜1.7): (1.5˜2.5): (3.5˜4.5).

12. The method, as recited in claim 11, wherein:
mass ratio of the ginseng, the peony, the poria, the licorice, the orange peel, the yam, the mint, the peach seed, the salvia and the penthorum chienese is (1.5, 2 or 2.5): (1.5, 2 or 2.5): (1.9, 2.4 or 2.9): (0.5, 1 or 1.5): (0.8, 1.2 or 1.7): (1.5, 2 or 2.5): (0.8, 1.2 or 1.7): (0.8, 1.2 or 1.7): (1.5, 2 or 2.5): (3.5, 4 or 4.5).

13. The method, as recited in claim 10, wherein:
in the methods for preparing the A) substance:
reflux extraction time is 2 h˜5 h;
a concentration of the aqueous solution of ethanol is 70%˜95% (according to volume fraction);
wherein in the methods for preparing the B) substance:
immersion time is 1 min˜60 min, the immersion time is preferably 1 min;
a distillation temperature is 90° C.˜100° C.;
wherein in the method for preparing the C) substance:
a boiling temperature is 90° C.˜100° C.; a boiling time is 1 h˜2 h;
a method for adding the absolute ethanol comprises adding the ethanol into the filtrate until a volume percentage of the ethanol in the filtrate is 40%;
fermentation temperature is 18° C.˜37° C.; fermentation time is 10 days to 180 days; a fermentation method comprises stirring while fermenting.

14. The method, as recited in claim 13, wherein:
in the methods for preparing the A) substance:
reflux extraction time is 2 h, 3 h or 5 h;
a concentration of the aqueous solution of ethanol is 70%, 90% or 95%;
wherein in the methods for preparing the B) substance:
immersion time is 1 min, 30 min or 60 min;
a distillation temperature is 90° C., 95° C. or 100° C.
wherein in the method for preparing the C) substance:
a boiling temperature is 90° C., 95° C. or 100° C.; a boiling time is 1 h or 2 h;
a method for adding the absolute ethanol comprises adding the ethanol into the filtrate until a volume percentage of the ethanol in the filtrate is 40%;
fermentation temperature is 18° C., 23° C. or 37° C.; fermentation time is 10 days, 15 days or 180 days; a fermentation method comprises stirring while fermenting.

15. The method, as claimed in claim 10, wherein:
the method for preparing the A) substance further comprises steps of:
removing the ethanol from the extract, and then concentrating and drying for obtaining a dried product, wherein the dried product is the ethanol extract of the ginseng and the salvia;
the method for preparing the C) substance further comprises steps of:
removing the ethanol from the supernatant after collecting the supernatant, filtering and collecting the filtrate, and then concentrating and drying for obtaining the dried product, wherein the dried product is the aqueous extract of the ginseng, the salvia, the mint, the orange peel, the peony, the poria, the licorice, the peach seed, the yam and the penthorum chienese;
the method for preparing the D) substance further comprises steps for crushing the fruits and vegetables into 40˜50 meshes size before the fermentation;
after the fermentation, the method for preparing the D) substance further comprises steps of: filtering the fermentation product, collecting the filtrate, ultrafiltering, collecting ultrafiltrate, wherein the ultrafiltrate is the fermentation conversion solution of fruits and vegetables.

16. The method, as claimed in claim 14, wherein:
the method for preparing the A) substance further comprises steps of:
removing the ethanol from the extract, and then concentrating and drying for obtaining a dried product, wherein the dried product is the ethanol extract of the ginseng and the salvia;
the method for preparing the C) substance further comprises steps of:
removing the ethanol from the supernatant after collecting the supernatant, filtering and collecting the filtrate, and then concentrating and drying for obtaining the dried product, wherein the dried product is the aqueous extract of the ginseng, the salvia, the mint, the orange peel, the peony, the poria, the licorice, the peach seed, the yam and the penthorum chienese;
the method for preparing the D) substance further comprises steps for crushing the fruits and vegetables into 40˜50 meshes size before the fermentation;
after the fermentation, the method for preparing the D) substance further comprises steps of: filtering the fermentation product, collecting the filtrate, ultrafiltering, collecting ultrafiltrate, wherein the ultrafiltrate is the fermentation conversion solution of fruits and vegetables.

17. The method, as recited in claim 10, wherein:
the fruits and vegetables are the mixture of the following 54 kinds of fruits and vegetables:
konjac, eggplant, asparagus, spinach, bean sprout, broccoli, cabbage, turnip, cucumber, pea, red pepper, celery, scallion, garlic, grape, grapefruit, watermelon, peach, orange, blueberry, orange, banana, lychee, bitter gourd, leek, pomegranate, dragon fruit, carrot, tomato, Chinese cabbage, celery, bell pepper, lettuce, pear, ginger, taro, bean, pumpkin, lotus root, cherry, kiwi, plum, strawberry, fig, kumquat, citrus, Southland pear, melon, cantaloupe, pawpaw, onion, mulberry, sugar beet and lemon;
wherein, each fruit or vegetable is of the same weight.

18. The method, as recited in claim 16, wherein:
the fruits and vegetables are the mixture of the following 54 kinds of fruits and vegetables:
konjac, eggplant, asparagus, spinach, bean sprout, broccoli, cabbage, turnip, cucumber, pea, red pepper, celery, scallion, garlic, grape, grapefruit, watermelon, peach, orange, blueberry, orange, banana, lychee, bitter gourd, leek, pomegranate, dragon fruit, carrot, tomato, Chinese cabbage, celery, bell pepper, lettuce, pear, ginger, taro, bean, pumpkin, lotus root, cherry, kiwi, plum, strawberry, fig, kumquat, citrus, Southland pear, melon, cantaloupe, pawpaw, onion, mulberry, sugar beet and lemon;
wherein, each fruit or vegetable is of the same weight.

19. The method, as claimed in claim 10, wherein:
in the method, every 1 L the mixture is prepared by: mixing (5˜50) g the ethanol extract of the ginseng and the salvia, (5˜50) g the volatile oil of the mint and the orange peel, (50˜200) g the aqueous extract of the ginseng, the salvia, the mint, the orange peel, the peony, the poria, the licorice, the peach seed, the yam and the penthorum chienese and the fermentation conversion solution of the fruits and vegetables, adding the fermentation conversion solution of fruits and vegetables for making up a volume to 1 L;
in the method for preparing the A) substance:
a ratio of the aqueous solution of the ethanol, the ginseng and the salvia is (1˜10) ml: 0.5 g: 0.5 g;
in the method for preparing the B) substance:
the ratio of the mint, the orange peel and the water is (1˜10) g: (1˜10) g: (1˜10) ml;
in the method for preparing the C) substance:
the ratio of the peony, the poria, the licorice, the peach seed, the yam, the penthorum chienese and the water is (1˜10) g: (1˜10) g: (1˜10) g: (1˜10) g: (1˜10) g: (1˜10) g: (10˜100) ml;
in the method for preparing the D) substance:
the ratio of the bacteria liquid of the Lactobacillus acidophilus, the bacteria liquid of the Bifidobactreium longum, the bacteria liquid of the Lactobacillus delbrueckii subsp bulgaricus, the bacteria liquid of the Streptococcus thermophilus, the fermentation conversion solution of the fruits and vegetables and water is (2000˜8000) ml: (2000˜8000) ml: (2000˜8000) ml: (2000˜8000) ml: (1000˜1500) kg: (1000˜1500) kg.

20. The method, as claimed in claim 19, wherein:
in the method, every 1 L the mixture is prepared by: mixing (5, 15 or 50) g the ethanol extract of the ginseng and the salvia, (5, 20 or 50) g the volatile oil of the mint and the orange peel, (50, 160 or 200) g the aqueous extract of the ginseng, the salvia, the mint, the orange peel, the peony, the poria, the licorice, the peach seed, the yam and the penthorum chienese and the fermentation conversion solution of the fruits and vegetables, adding the fermentation conversion solution of fruits and vegetables for making up a volume to 1 L;
in the method for preparing the A) substance:
a ratio of the aqueous solution of the ginseng and the salvia is (1, 5 or 10) ml: 0.5 g: 0.5 g;
in the method for preparing the B) substance:
the ratio of the mint, the orange peel and the water is (1, 5 or 10) g: (1, 5 or 10) g: (1, 5 or 10) ml;
in the method for preparing the C) substance:
the ratio of the peony, the poria, the licorice, the peach seed, the yam, the penthorum chienese and the water is (1, 2 or 10) g: (1, 2.4 or 10) g: (1, 5 or 10) g: (1, 1.2 or 10) g: (1, 2 or 10) g: (1, 4 or 10) g: (50, 60 or 100) ml;
in the method for preparing the D) substance:
the ratio of the bacteria liquid of the Lactobacillus acidophilus, the bacteria liquid of the Bifidobactreium longum, the bacteria liquid of the Lactobacillus delbrueckii subsp bulgaricus, the bacteria liquid of the Streptococcus thermophilus, the fermentation conversion solution of the fruits and vegetables and the water is (2000, 5000 or 8000) ml: (2000, 5000 or 8000) ml: (2000, 5000 or 8000) ml: (2000, 5000 or 8000) ml: (1000, 1200 or 1500) kg: (1000, 1200 or 1500) kg.

21. The method, as recited in claim 10, wherein:
a method for preparing the bacteria liquid of the Lactobacillus acidophilus comprises: fermenting and growing Lactobacillus acidophilus for obtaining the fermentation production, wherein the fermentation production is the bacteria liquid of the Lactobacillus acidophilus; wherein the fermentation temperature is 20° C.˜41° C., the fermentation time is 15 h˜36 h;
a method for preparing the bacteria liquid of the Bifidobactreium longum comprises: fermenting and growing Bifidobactreium longum for obtaining the fermentation production, wherein the fermentation production is the bacteria liquid of the Bifidobactreium longum; wherein the fermentation temperature is 20° C.˜41° C., the fermentation time is 15 h˜36 h;
a method for preparing the bacteria liquid of the Lactobacillus delbrueckii subsp bulgaricus comprises: fermenting and growing Lactobacillus delbrueckii subsp bulgaricus for obtaining the fermentation production, wherein the fermentation production is the bacteria liquid of the Lactobacillus delbrueckii subsp bulgaricus; wherein the fermentation temperature is 20° C.˜41° C., the fermentation time is 15 h˜36 h;
a method for preparing the bacteria liquid of the Streptococcus thermophilus comprises: fermenting and growing Streptococcus thermophilus for obtaining the fermentation production, wherein the fermentation production is the bacteria liquid of the Streptococcus thermophilus; wherein the fermentation temperature is 20° C.˜41° C., the fermentation time is 15 h˜36 h.

22. The method, as recited in claim 19, wherein:
a method for preparing the bacteria liquid of the Lactobacillus acidophilus comprises: fermenting and growing Lactobacillus acidophilus for obtaining the fermentation production, wherein the fermentation production is the bacteria liquid of the Lactobacillus acidophilus; wherein the fermentation temperature is 20° C.˜41° C., the fermentation time is 15 h˜36 h;
a method for preparing the bacteria liquid of the Bifidobactreium longum comprises: fermenting and growing Bifidobactreium longum for obtaining the fermentation production, wherein the fermentation production is the bacteria liquid of the Bifidobactreium longum; wherein the fermentation temperature is 20° C.˜41° C., the fermentation time is 15 h˜36 h;
a method for preparing the bacteria liquid of the Lactobacillus delbrueckii subsp bulgaricus comprises: fermenting and growing Lactobacillus delbrueckii subsp bulgaricus for obtaining the fermentation production, wherein the fermentation production is the bacteria liquid of the Lactobacillus delbrueckii subsp bulgaricus; wherein the fermentation temperature is 20° C.˜41° C., the fermentation time is 15 h˜36 h;
a method for preparing the bacteria liquid of the Streptococcus thermophilus comprises: fermenting and growing Streptococcus thermophilus for obtaining the fermentation production, wherein the fermentation production is the bacteria liquid of the Streptococcus thermophilus; wherein the fermentation temperature is 20° C.˜41° C., the fermentation time is 15 h˜36 h.

23. The method, as recited in claim 22, wherein:
a method for preparing the bacteria liquid of the Lactobacillus acidophilus comprises: fermenting and growing Lactobacillus acidophilus for obtaining the fermentation production, wherein the fermentation production is the bacteria liquid of the Lactobacillus acidophilus; wherein the fermentation temperature is 20° C., 37° C. or 41° C., the fermentation time is 15 h, 16 h or 36 h;
a method for preparing the bacteria liquid of the Bifidobactreium longum comprises: fermenting and growing Bifidobactreium longum for obtaining the fermentation production, wherein the fermentation production is the bacteria liquid of the Bifidobactreium longum; wherein the fermentation temperature is 20° C., 37° C. or 41° C., the fermentation time is 15 h, 16 h or 36 h;
a method for preparing the bacteria liquid of the Lactobacillus delbrueckii subsp bulgaricus comprises: fermenting and growing Lactobacillus delbrueckii subsp bulgaricus for obtaining the fermentation production, wherein the fermentation production is the bacteria liquid of the Lactobacillus delbrueckii subsp bulgaricus; wherein the fermentation temperature is 20° C., 37° C. or 41° C., the fermentation time is 15 h, 16 h or 36 h;
a method for preparing the bacteria liquid of the Streptococcus thermophilus comprises: fermenting and growing Streptococcus thermophilus for obtaining the fermentation production, wherein the fermentation production is the bacteria liquid of the Streptococcus thermophilus; wherein the fermentation temperature is 20° C., 37° C. or 41° C., the fermentation time is 15 h, 16 h or 36 h.

24. The method, as recited in claim 10, wherein:
the Lactobacillus acidophilus is the Lactobacillus acidophilus CGMCC (China General Microbiological Culture Collection) 1.1854, the Bifidobactreium longum is the Bifidobactreium longum CGMCC 1.2186, the Lactobacillus delbrueckii subsp bulgaricus is the Lactobacillus delbrueckii subsp bulgaricus CGMCC 1.1480, the Streptococcus thermophilus is the Streptococcus thermophilus CGMCC 1.2471.

25. The method, as recited in claim 21, wherein:
the Lactobacillus acidophilus is the Lactobacillus acidophilus CGMCC (China General Microbiological Culture Collection) 1.1854, the Bifidobactreium longum is the Bifidobactreium longum CGMCC 1.2186, the Lactobacillus delbrueckii subsp bulgaricus is the Lactobacillus delbrueckii subsp bulgaricus CGMCC 1.1480, the Streptococcus thermophilus is the Streptococcus thermophilus CGMCC 1.2471.

26. The method, as recited in claim 22, wherein:
the Lactobacillus acidophilus is the Lactobacillus acidophilus CGMCC (China General Microbiological Culture Collection) 1.1854, the Bifidobactreium longum is the Bifidobactreium longum CGMCC 1.2186, the Lactobacillus delbrueckii subsp bulgaricus is the Lactobacillus delbrueckii subsp bulgaricus CGMCC 1.1480, the Streptococcus thermophilus is the Streptococcus thermophilus CGMCC 1.2471.

27. The method, as recited in claim 23, wherein:
the Lactobacillus acidophilus is the Lactobacillus acidophilus CGMCC (China General Microbiological Culture Collection) 1.1854, the Bifidobactreium longum is the Bifidobactreium longum CGMCC 1.2186, the Lactobacillus delbrueckii subsp bulgaricus is the Lactobacillus delbrueckii subsp bulgaricus CGMCC 1.1480, the Streptococcus thermophilus is the Streptococcus thermophilus CGMCC 1.2471.

28. A pharmaceutical composition for treating melasma prepared by the method recited in claim 10.

29. A pharmaceutical composition for treating melasma prepared by the method recited in claim 27.
